(19) United States
(12) Patent Application Publication
(10) Pub. No.: US 2016/0120500 A1
(43) Pub. Date: May 5, 2016

(54) FETAL HEART RATE MONITORING ASSEMBLY

(71) Applicant: LAERDAL GLOBAL HEALTH AS, Stavanger (NO)

(72) Inventors: Helge MYKLEBUST, Stavanger (NO); Helge FOSSAN, Stavanger (NO); Joar EILEVSTJØNN, Sandnes (NO); Naci Cenk AYTEKIN, Stavanger (NO)

(73) Assignee: Laerdal Global Health AS, Stavanger (NO)

(21) Appl. No.: 14/898,799

(22) PCT Filed: Jun. 26, 2014
(86) PCT No.: PCT/NO2014/050115

§ 371 Date: Dec. 16, 2015

(30) Foreign Application Priority Data
Jul. 12, 2013 (NO) 20130969

(51) Int. Cl. A61B 8/08; A61B 5/00; A61B 5/11; A61B 5/024; A61B 8/02; A61B 8/00

(52) U.S. Cl. CPC A61B 8/0866; A61B 8/02; A61B 8/56; A61B 8/4227; A61B 5/11; A61B 5/02438; A61B 8/5276; A61B 8/464; A61B 5/7271; A61B 2562/0219

(57) ABSTRACT

A fetal heart rate measuring assembly (1) comprising an ultrasound transducer (71) adapted to perform fetal heart rate measurements when attached to the abdomen of a pregnant woman, a logic unit (31) connected to the ultrasound transducer (71), a battery (39), and a measurement presentation unit (35), wherein the entire assembly (1) is adapted to be attached to said pregnant woman. The assembly further comprises an accelerometer (73) adapted to sense movement of the pregnant woman.

The present invention relates to a monitoring assembly adapted to monitor heart rates of a fetus. In particular the monitoring assembly is adapted for such monitoring during labor in low resource environments, typically maternity units having few midwifes and small resources.

### BACKGROUND

Each year approximately 136 million babies are born globally, and it is estimated that about 90% make the transition from intrauterine to extra uterine life without any intervention. The remaining 13.6 million newborns are delivered with absent or poor respiratory effort and need some degree of support to achieve cardiopulmonary stability. Between 3-6% need assisted positive pressure ventilation, and less than 1% require advanced resuscitation including intubation, chest compressions, and medications.

Intrapartum hypoxia (often equated with birth asphyxia) is estimated to account for about two million perinatal deaths annually including intrapartum stillbirths and early neonatal deaths, with 98-99% of the burden in low and middle income countries. An additional one million of the surviving infants develop neurocognitive problems such as cerebral palsy and learning difficulties.

The incidence of intrapartum-related stillbirths and neonatal deaths has remained essentially unchanged over the past 15 years. The lack of impact on neonatal mortality rates the first day of life is a major concern.

International patent application publication WO2010098767 describes a monitoring system having a monitoring device with an accelerometer that senses fetal movements, such as a kick, of a fetus in the womb of a woman. Signals from the accelerometer are identified by a signal identifier to separate fetal movements from other movements. The system is adapted to monitor fetal heart rate as well as the activity of the mother. In one embodiment, the accelerometer is held in place on the mother by means of a belt. In another embodiment an adhesive is used. Moreover, in one embodiment a signal indicative of the fetal heart rate is used to facilitate placement of the accelerometer. The system may also comprise an ultrasound transducer that senses signals indicative of the fetal heart rate.

Patent application publication US20110160591 describes a fetal heart rate monitor having an ultrasound probe positioned on the abdomen of the mother. The probe is strapped onto the mother with a strap and connected to a table-mounted monitor device through a cable or wireless.

U.S. Pat. No. 4,809,702 describes an apparatus for recording of fetus movement. It comprises an ultrasonic transducer and a display which is arranged in a case. The case is strapped onto the mother's abdominal skin by means of a strap. The apparatus can also be used for detection of movement of volumes other than the fetuses, such as heartbeat of the mother.

### The Invention

According to the present invention there is provided a fetal heart rate (FHR) measuring assembly comprising an ultrasound transducer adapted to perform fetal heart rate measurements when attached to the abdomen of a pregnant woman. The assembly comprises a logic unit connected to the ultrasound transducer, a battery, and a measurement presentation unit. The entire assembly is adapted to be attached to the pregnant woman. According to the invention the assembly further comprises an accelerometer which is adapted to sense movement of the pregnant woman.

In one embodiment, the accelerometer is adapted to sense both movement and orientation of the pregnant woman with respect to gravity.

The measurement presentation unit is a unit adapted for communicating fetal heart rate measurements to a person, typically a midwife or other medical personnel, or to the mother. It may for instance be a display or a wireless communication arrangement.

Such an assembly makes the mother mobile while maintaining monitoring of the fetal heart rate, since the ultrasound transducer is not wired to a stationary component. Moreover, the mother may herself monitor the FHR and hence relieve the work of the midwifes. Preferably the assembly may have an alarm function in order to alert the mother or midwife if the FHR is critical.

In one embodiment, the logic unit is adapted to discard fetal heart rate measurements performed during excessive movements, and/or perform fetal heart rate measurements only during periods having permitted movements.

Permitted movements are movements below a predetermined threshold value. Such threshold value will typically be a value which will not excessively disturb the FHR measurement. Excessive movements may typically occur during contractions, when the mother turns in her bed, or when she is up walking.

The assembly may advantageously comprises a timekeeping unit (a clock) and a memory unit, and be adapted to record fetal heart rate measurement results in the memory unit. The assembly can then allocate times at which the measurements took place and the position of the pregnant woman, to the results. The fetal heart rate measurement results can then be presented by the assembly with the allocated time and position by means of the measurement presentation unit.

Advantageously the assembly can comprise a proximity sensor functionally connected to the logic unit. The logic unit can then be adapted to initiate a fetal heart rate measurement upon receiving an initiation signal from the proximity sensor.

The proximity sensor is adapted to sense the appearance of a proximate object, such as the midwife's hand.

In a particular embodiment, such an initiation signal comprises a plurality of succeeding separate proximity measurements according to a predetermined initiation signal pattern.

An initiation signal pattern is a predetermined pattern of outputs from the proximity sensor. For instance, the logic unit may be programmed to interpret three rapidly (e.g. within one second) succeeding outputs from the proximity sensor as a valid initiation signal.

As a result of the proximity sensor, the midwife (or other person) does not need to touch the assembly in order to initiate a FHR measurement.

Moreover, the assembly may further comprise a light sensor functionally connected to the logic unit, wherein the logic unit is adapted to discard initiation signals from the proximity sensor if the light sensor senses light values below a predetermined value. Such an embodiment would contribute in reducing accidentally initiations of FHR measurements.

The ultrasound transducer can advantageously be arranged in a transducer housing, while the battery and the logic unit are arranged in a main housing. The ultrasound transducer is then connected to the logic unit through a wire extending between the transducer housing and the main housing. Preferably the accelerometer has three axis (X, Y, Z) and is arranged as part of the transducer housing.

The assembly according to the invention may comprise an electrocardiography heart rate unit which is adapted to measure the heart rate of the pregnant mother, and the logic unit can be adapted to discriminate between the measured fetal heart rate and the measured heart rate of the pregnant mother. Preferably the heart rate is detected by using dry electrodes.

Thus by employing the measured values of the mother's heart rate, the assembly can be adapted to avoid taking the mother's heart rate as the fetal heart rate. For instance, the assembly, typically the logic unit, will compare the heart rate value with the heart rate value from the fetal heart rate measuring assembly, and if the heart rate values overlap, the assembly will indicate on the display that the fetal heart rate has not been found.

The assembly may comprise a main housing connected to a harness adapted to be suspended about the neck of the pregnant mother, and the main housing can comprise a second display and the measurement presentation unit which is a first display.

In such an embodiment, the second display may face towards the face of the pregnant mother when the main housing is carried around the neck with the harness. The second display is in this manner adapted to present information to the pregnant mother.

Moreover, in this embodiment the first display may face away from the face of the pregnant mother when the main housing is carried around the neck with the harness.

In one embodiment, the first display may face downwards, hence out of sight for the pregnant mother.

### EXAMPLE OF EMBODIMENT

Having described the present invention in general terms above, a more detailed example of embodiment will be described in the following with reference to the drawings, in which

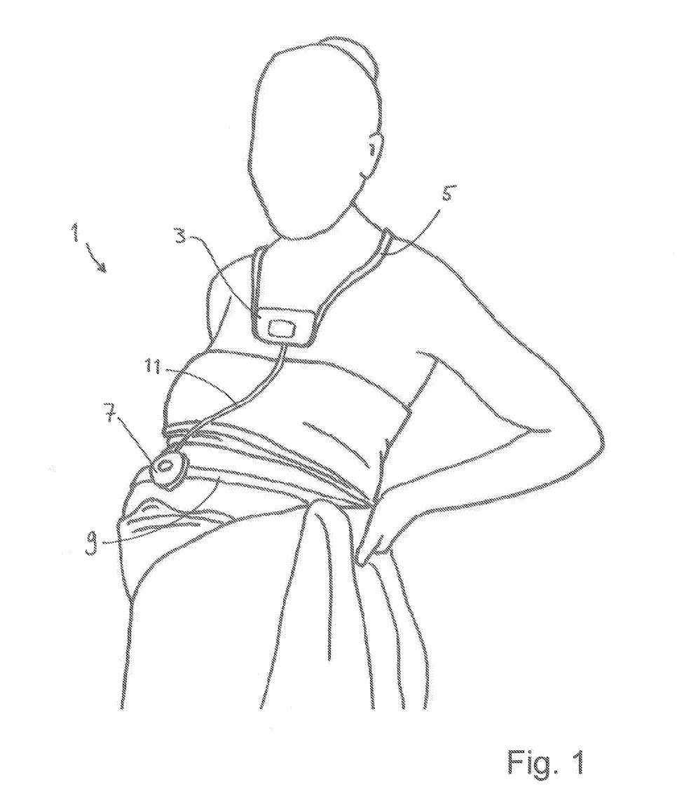

FIG. 1 illustrates a FHR measuring assembly according to the invention being carried by a pregnant woman;

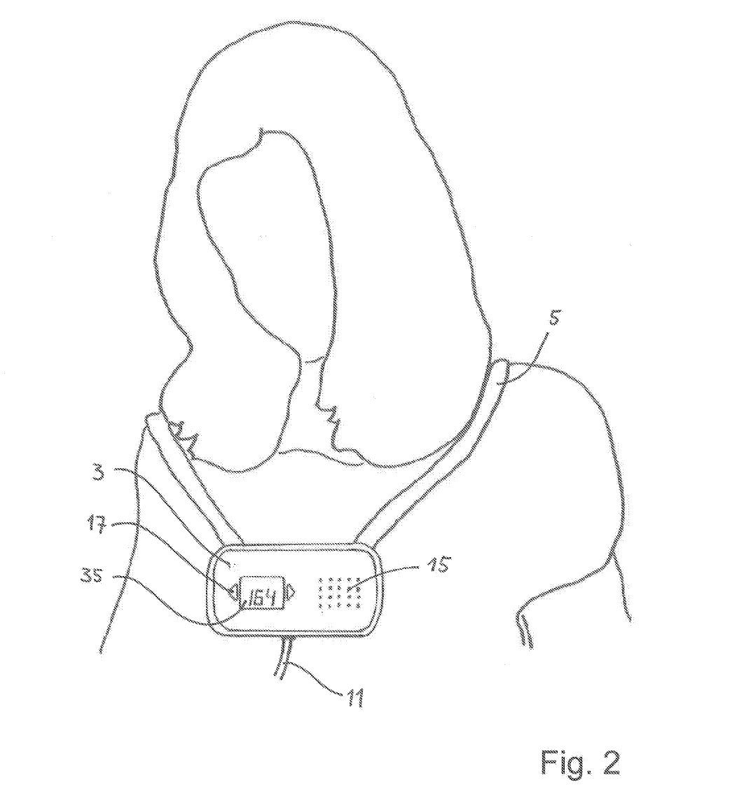

FIG. 2 is an enlarged view of parts of the assembly carried by a pregnant woman;

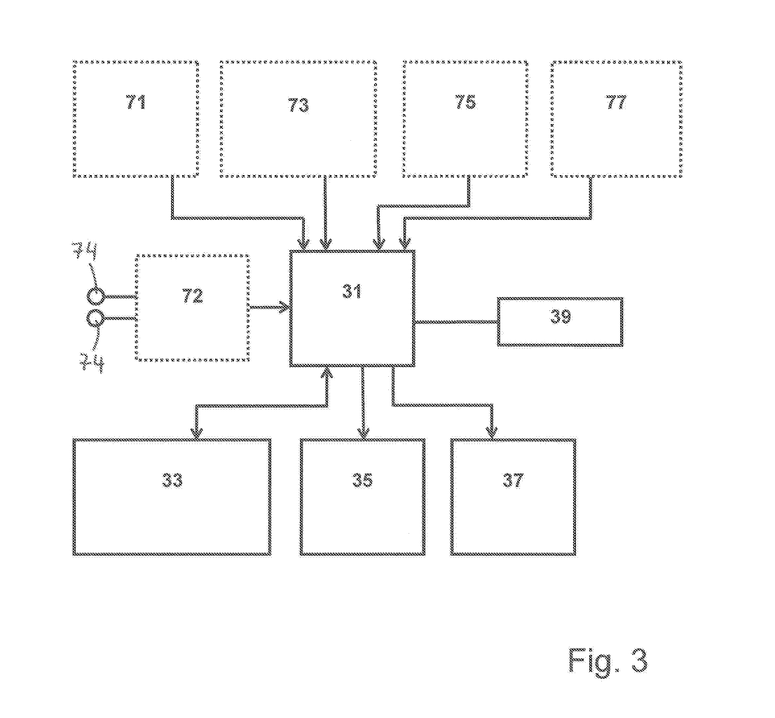

FIG. 3 is a schematic view of various components of the assembly and their functional connections;

FIG. 4 is an enlarged perspective view of a transducer housing provided with a proximity sensor which is manipulated by a hand;

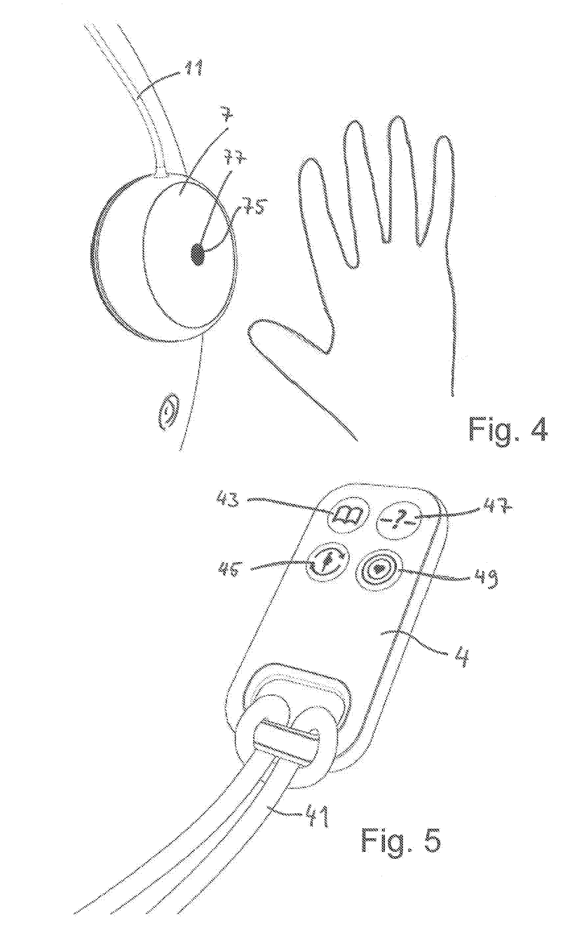

FIG. 5 is a perspective view of a remote control adapted to operate the FHR measuring assembly;

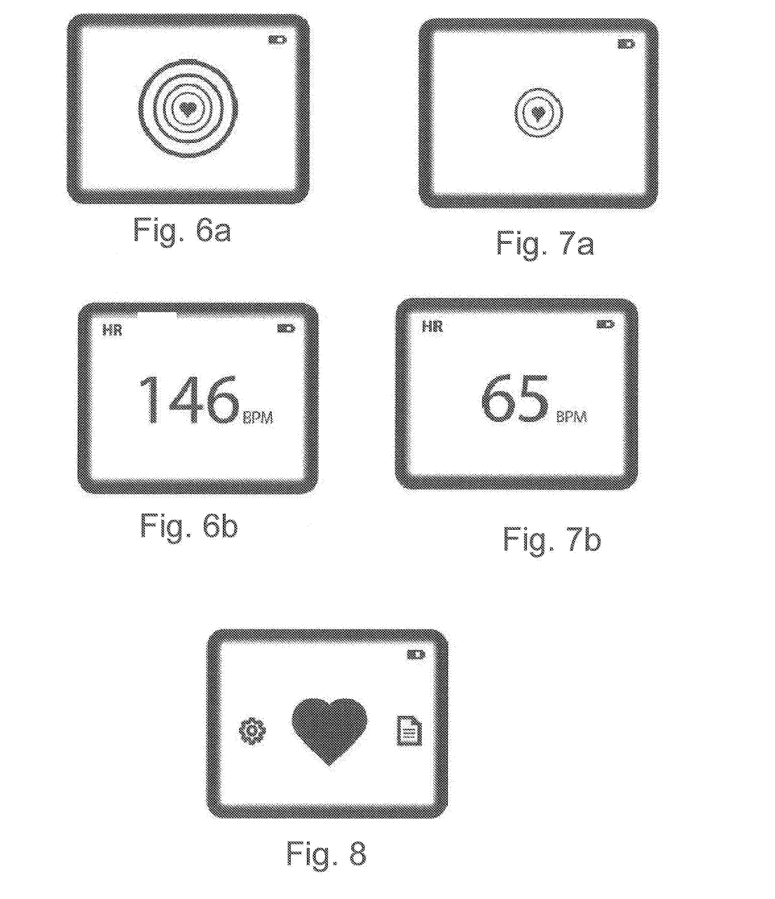

FIG. 6a and FIG. 6b show two ways of presenting a measured FHR in a display;

FIG. 7a and FIG. 7b show the two ways of presenting a measured FHR corresponding to FIG. 6a and FIG. 6b, however presenting a lower FHR;

FIG. 8 shows a menu in a display;

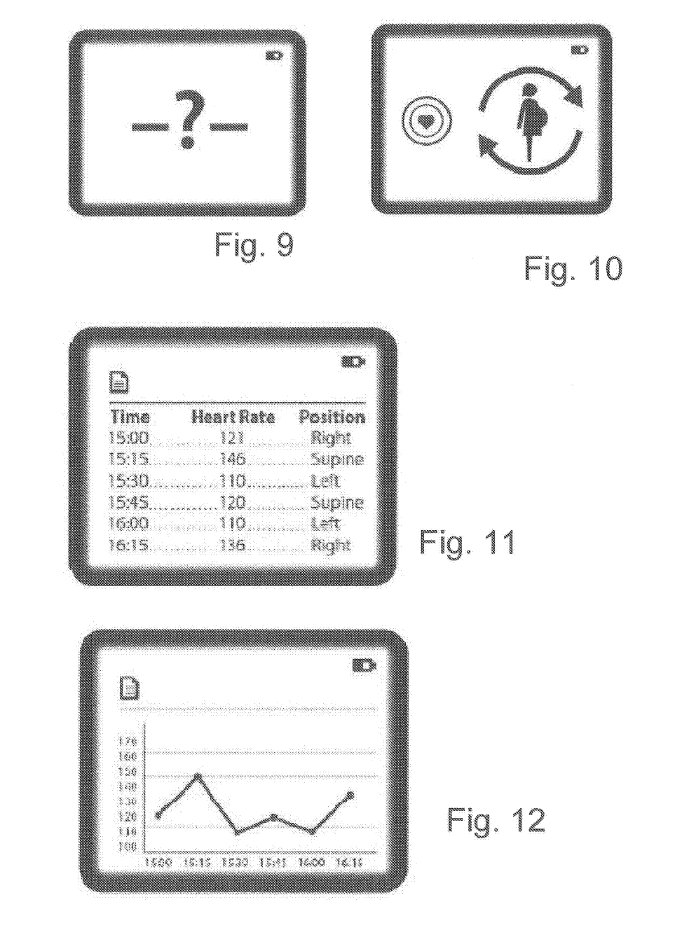

FIG. 9 depicts a display with a symbol indicating that the assembly is not able to detect a FHR;

FIG. 10 shows an example of the display in a situation where the measured fetal heart rate is too low;

FIG. 11 illustrates an example of presentation of a data log in a display;

FIG. 12 illustrates another example of presentation of a data log in a display;

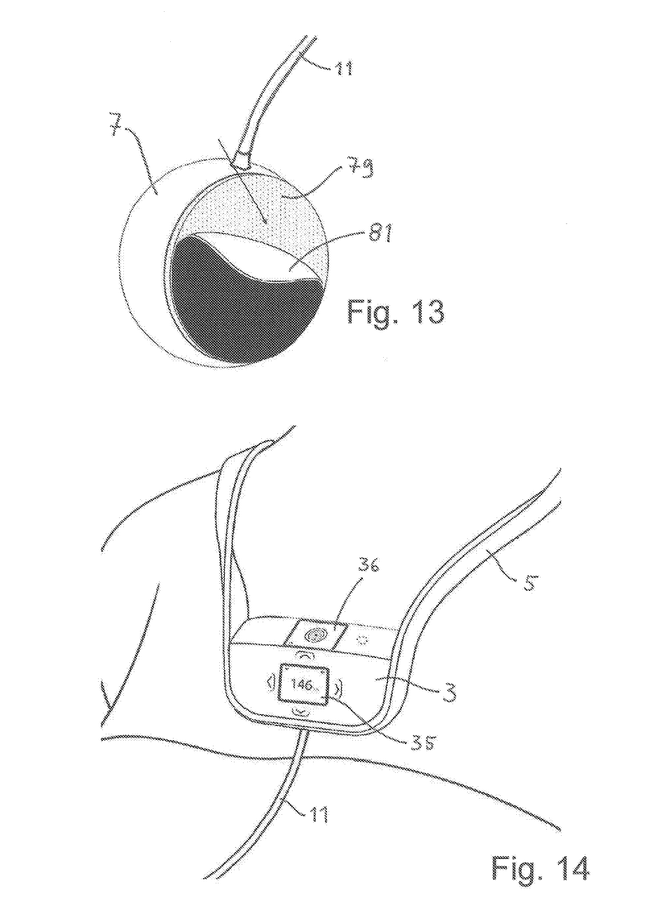

FIG. 13 is a perspective view showing a transducer housing provided with an adhesive hydrogel film; and

FIG. 14 is an enlarged perspective view showing an alternative embodiment of a main housing.

FIG. 1 illustrates a pregnant woman having a fetal heart rate measuring assembly 1 arranged to her. A main housing 3 is loosely hung around the neck of the woman by means of a harness 5. A transducer housing 7 is strapped onto the abdomen of the woman by means of a strap 9. A wire 11 connects the main housing 3 to the transducer housing 7.

FIG. 2 illustrates another main housing 3 arranged to a woman. The main housing 3 of this embodiment has measurement presentation unit in the form of a display 35 showing the current or last measured heart beat of a fetus. It also comprises an audio communication means 15, as well as control buttons 17 for controlling the function of the heart rate measuring assembly 1.

FIG. 3 illustrates a schematic view of some of the components of the fetal heart rate assembly 1. An ultrasound transducer 71 and an accelerometer 73 are arranged in the transducer housing 7 (indicated with dashed lines). The transducer housing 7 also comprises a light sensor 75 and a proximity sensor 77. Through the wire 11, the ultrasound transducer 71, the accelerometer 73, the light sensor 75 and the proximity sensor 77 are connected to a logic unit 31 which is arranged within the main housing 3. The logic unit 31 will typically be a programmed microcontroller.

The transducer housing 7 may also comprise an electrocardiography (ECG)/heart rate unit 72 which by ECG electrodes 74 on the transducer housing 7 or on an attachment belt enables measurement of the heart rate of the mother. By measuring the heart rate of the mother the assembly 1 can discriminate between the fetal heart rate and the mother's heart rate. This will ensure proper orientation of the ultrasound transducer 71 so that a misinterpretation of the mother's heart rate as the fetal heart rate is avoided.

As indicated with the arrows, the ultrasound transducer 71, accelerometer 73, light sensor 75 and proximity sensor 77 are all adapted to provide input to the logic unit 31.

Output data from the logic unit 31 comprises data delivered to a wireless communication unit 33, a display 35, and an alert unit 37. These components are all arranged in the main housing 3. Also arranged in the main housing 35 is a battery 39.

Not shown in the schematic drawing of FIG. 3 is a battery charging circuitry, a timekeeping unit (clock), a memory unit, as well as an additional wire interface, such as a USB interface. The timekeeping unit and the memory unit may advantageously be parts of the logic unit 31.

By means of the accelerometer 73, the logic unit 31 is able to discard heart rate measurements which have taken place during excessive movement of the pregnant woman. If the logic unit 31 detects such excessive movements during a heart rate measurement, it may initiate another measurement in order to provide a heart rate measurement which has occurred during sufficiently good measuring conditions.

The various components introduced with reference to FIG. 3 will be described in more detail under reference to the succeeding drawings.

FIG. 4 illustrates the transducer housing 7 attached to the abdomen of a pregnant woman. In this embodiment the proximity sensor 77 is arranged in an outwardly facing face of the transducer housing 7. With her hand, a midwife or the mother herself, or another person, may trigger a fetal heart rate measurement by moving her hand in front of the proximity sensor 77. The proximity sensor 77 will upon such movement, send a signal to the logic unit 31 in the main housing 3 through the wire 11. When receiving the signal, the logic unit will initiate a measurement of the fetal heart rate by means of the ultrasound transducer 71 which also is arranged in the transducer housing 7.

Further, by operating the ultrasound transducer 71 through the wire 11, the logic unit 31 will receive input from the transducer 71 and thereby obtain the fetus heart rate. The logic unit 31 transmits the heart rate value to the display 35. In this way, a quick initiation and performance of a fetus heart rate measurement has been accomplished. As will be appreciated by the skilled person, with this setup a midwife does not need to touch the fetal heart rate assembly 1 in order to obtain a measurement and a contamination is thus avoided.

In an advantageous embodiment, the transducer housing 7 also comprises a light sensor 75 by means of which the logic unit 31 detects whether or not the transducer housing 7 is covered, such as by a duvet or blanket. This will contribute in avoiding unintended initiations, as the logic unit 31 will discard signals from the proximity sensor 77 if the transducer housing 7 is covered. The light sensor 75 may be arranged together with the proximity sensor 77.

In order to avoid unintended initiations, as an alternative or an additional measure to the light sensor 75 described above, the logic unit 31 may be programmed in such way that only a specific detection pattern from the proximity sensor 77 will trigger a measurement initiation. For instance, the logic unit 31 may only initiate a heart rate measurement when receiving three signals from the proximity sensor 77 within one second. A single unintended movement in front of the proximity sensor 77 would then not trigger a measurement.

The proximity sensor 77 could also be arranged on the main housing.

FIG. 5 illustrates a remote control 4 by means of which a medical person, typically a midwife, can control the functions of the fetal heart rate measuring assembly 1.

A lace 41 is attached to the remote control 4 so that the medical person can hang the remote control 4 around her neck.

When pressing the Training mode′ button 43 on the remote control 4, the fetal heart rate measuring assembly 1 will operate in training mode. During training mode, the instructor, typically a midwife, can simulate various functions of the fetal heart rate measuring assembly 1 to train medical staff. These functions may include “patient position change”, “loss of heart rate” and “toggle between low heart rate and normal heart rate”.

When the “Patient position change” button 45 is pressed, the fetal heart rate measuring assembly 1 can simulate a random position change of the pregnant mother on the bed. In a real scenario, a mother position change may interfere with the fetal heart rate reading due to a possible relocation of the fetus or placenta inside the stomach. This relationship between the fetal heart rate reading and the position of the mother is important and needs to be logged to understand which position of the mother provides the best fetal heart rate reading. Therefore a “Loss of heart rate” button 47 and a “Toggle between low heart rate and normal heart rate” button 49 are used to simulate which mother position is good in relation to the fetal heart rate. These buttons may also be used to simulate an emergency situation of the baby.

The “Loss of heart rate” 49 button is illustrated by a question mark on the remote control 4. By pressing on the “Loss of heart rate” button 49, the display 35 (FIG. 2) shows a question mark which means that the student needs to readjust the location of the ultrasound probe on stomach of the pregnant mother and search for consistent fetal heart rate. By continuing this simulation repetitively, the instructor can also create a log on the device to teach the student, which position of the mother is better for a reading of a healthy and consistent fetal heart rate.

The “Toggle between low heart rate and normal heart rate” button 49 simulates a low heart rate reading and a healthy heart rate reading on the display. Each press to the button toggles between low and healthy fetal heart rate. The “Toggle between low heart rate and normal heart rate” button 49 can be used to simulate either a complication with the fetus or a bad position of the mother for the ultrasound probe to get a healthy and consistent reading of the fetal heart rate.

In one embodiment the remote control 4 is adapted to communicate wirelessly with the closest fetal heart rate assembly 1, in a situation where a plurality of such assemblies is present. Hence, for instance a midwife can carry one remote control 4 while visiting one pregnant mother after the other and control only the fetal heart rate assembly 1 pertaining to the patient she is visiting.

FIG. 6a to FIG. 7a show two different ways of communicating a fetal heart rate on the display 35. FIG. 6a illustrates a graphic symbol which indicates a strong, healthy heart rate within a predetermined heart rate region. FIG. 6b shows an alternative to the same heart rate as FIG. 6a by displaying the heart rate itself numerically. In this case the measured heart rate is 146 beats per minute. Hence, the number 146 is within the heart beat region depicted in FIG. 6a.

Correspondingly FIGS. 7a and 7b show a measured heart rate value in these two alternative ways. However, in these drawings the display shows a too low heart rate. FIG. 7b shows a measured heart rate of 65 beats per minute, while FIG. 7a illustrates the heart beat region containing the value of 65.

In a situation where the measured fetal heart beat is below a predetermined threshold value, the fetal heart rate measuring assembly 1 can be adapted to trigger an alarm signal, such as with the alert unit 37 schematically depicted in FIG. 3. Such an alarm signal can for instance be an audio warning signal emitted through an audio communication means 15 (loudspeaker), a wireless alarm signal sent to a receiver carried by or otherwise interfacing with a medical person. A wireless signal would typically be generated with the wireless communication unit 33.

FIG. 8 depicts a display that displays a menu for operation of the fetal heart rate assembly 1 according to the invention. The display has control buttons 17, such as arranged on a touch screen or physical buttons as shown in FIG. 2, that allow the user to navigate on the display 35. By navigating down or up, the picture of FIG. 8 is shown. From here, navigating left brings forward a screen that displays the settings of the system, and by navigating right the display presents a data log. FIG. 11 and FIG. 12 illustrate two examples of a displayed data log. This will be discussed below.

FIG. 9 shows an example of the display 35 in a situation where the fetal heart rate assembly 1 is not able to detect a fetal heart rate. The display 35 communicates in this manner that the transducer housing 7 should be repositioned in order to obtain the heart rate measurement.

FIG. 10 shows an example of the display 35 in a situation where the measured fetal heart rate is too low. On the left hand side of the screen in FIG. 10, the symbol indicating too low heart rate is shown, corresponding to the symbol shown in FIG. 7a. On the right hand side of the screen a re-position symbol is shown, which communicates to the pregnant mother or e.g. the midwife that the mother should change her position. For instance, she should move from lying on her left side to her right side.

As shown in the schematic illustration in FIG. 3, the fetal heart rate assembly 1 can also comprise an accelerometer 73. The accelerometer 73 is arranged within the transducer housing 7 and is thus fixed to the pregnant mother. By means of input from the accelerometer 73, the logic unit 31 is adapted to detect movements of the mother as well as position with respect to gravity. Moreover, with a clock and memory unit (not depicted in FIG. 3) in the logic unit 31, it is able to allocate time (T) as well as the position (P) of the pregnant woman to the heart rate measurements. An example of this is shown in FIG. 11, which illustrates the display 35 showing a set of heart rate measurements, along with the time of measurements and the position of the mother at the respective measurements. As is appreciated from the example of FIG. 11, the fetal heart rate measurement assembly 1 has performed a heart rate measurement every 15 minutes and shows on the display 35 the result of six succeeding measurements. By studying the information of the display example of FIG. 11, the mother herself or the midwife, for instance, will appreciate that the heart rate of the fetus is highest when the woman lies on her right side. When lying on her back (supine) the heart rate is lower, while the lowest measurements have occurred when she is lying on her left side.

Although the fetal heart rate assembly 1 advantageously will be programmed for performing heart rate measurements at given intervals, e.g. every 5, 10, or 15 minutes, it is preferably also able to perform a measurement on demand. For instance, after having repositioned the pregnant woman, one may want to perform a measurement at once without waiting for the next scheduled measurement. As described above with reference to FIG. 4, such a non-scheduled measurement may be initiated by manipulating the proximity sensor 77. One can also imagine that such a measurement can be initiated with the remote control 4 shown in FIG. 5.

FIG. 12 depicts the display 35 while showing a graphic representation of the same measurements as in the example of FIG. 11, however without information about the position of the pregnant mother. A graphic representation may make it easier to quickly spot a rising or falling trend, in lieu of comparing the numbers of the example of FIG. 11.

FIG. 13 depicts the reverse side of a transducer housing 7 without a strap 9 for attachment to the pregnant mother. In stead of the strap 9, a hydrogel film 79 is applied onto the reverse side of the transducer housing 7. It is well known to use hydrogel to ensure electric coupling between the electrodes of a defibrillator and a patient chest. In the embodiment shown in FIG. 13, however, a hydrogel film is used to provide both attachment of the transducer housing 7 to the skin of the pregnant mother, as well as acoustic coupling between the ultrasound transducer 71 and the abdomen of the mother. By using hydrogel in stead of the strap 9, it also becomes easier to reposition the transducer housing 7. In order to protect the hydrogel film 79 when the transducer housing 7 is not in use, a removable protective sheet 81 is applied to the hydrogel film 79.

FIG. 14 shows an embodiment where the main housing 3 is provided with two displays. A first display 35 corresponds to the display shown in FIG. 2, presenting a measured fetal heart rate value numerically. In addition the main housing 3 has a second display 36 which is adapted to show information to the pregnant mother that carries the main housing 3. In this embodiment the fetal heart rate value is graphically presented, as discussed above with reference to FIG. 6a and FIG. 7a.

In an embodiment alternative to the one shown in FIG. 14, the first display 35 may be arranged facing down, i.e. arranged on the lower side of the main housing 3.

1. A fetal heart rate measuring assembly comprising an ultrasound transducer adapted to perform fetal heart rate measurements when attached to the abdomen of a pregnant woman, a logic unit connected to the ultrasound transducer, a battery, and a measurement presentation unit, wherein the entire assembly is adapted to be attached to said pregnant woman, wherein it further comprises an accelerometer adapted to sense movement of the pregnant woman, wherein the accelerometer is adapted to sense orientation of the pregnant woman with respect to gravity.

2. The fetal heart rate measuring assembly according to claim 1, wherein the fetal heart rate measuring assembly comprises an electrocardiography heart rate unit adapted to measure the heart rate of the pregnant woman, connected to electrocardiography electrodes, wherein the electrocardiography electrodes are dry electrodes and are arranged on the transducer housing.

3. The fetal heart rate measuring assembly according to claim 1, wherein the logic unit is adapted to perform at least one of the following:
i) discard fetal heart rate measurements performed during excessive movements; and
ii) perform said fetal heart rate measurements only during periods having permitted movements.

4. The fetal heart rate measuring assembly according to claim 3, wherein the fetal heart rate measuring assembly comprises a timekeeping unit and a memory unit, and that it is adapted to record fetal heart rate measurement results in the memory unit, and allocate times (T) at which the measurements took place and orientation of pregnant woman to the results, and present the fetal heart rate measurement results with the allocated time (T) and orientation with the measurement presentation unit.

5. The fetal heart rate measuring assembly according to claim 1, wherein the fetal heart rate measuring assembly comprises a proximity sensor connected to the logic unit, wherein the logic unit is adapted to initiate a fetal heart rate measurement upon receiving an initiation signal from the proximity sensor.

6. The fetal heart rate measuring assembly according to claim 5, wherein the initiation signal comprises a plurality of succeeding separate proximity measurements according to a predetermined initiation signal pattern.

7. The fetal heart rate measuring assembly according to claim 5, wherein the fetal heart rate measuring assembly further comprises a light sensor connected to the logic unit, wherein the logic unit is adapted to discard initiation signals from the proximity sensor if the light sensor senses light values below a predetermined value.

8. The fetal heart rate measuring assembly according to claim 1, wherein the ultrasound transducer is arranged in a transducer housing, and that the battery and the logic unit are arranged in a main housing, wherein the ultrasound transducer is connected to the logic unit through a wire extending between the transducer housing and the main housing.

9. The fetal heart rate measuring assembly according to claim 2, wherein the logic unit is adapted to discriminate between the measured fetal heart rate and the measured heart rate of the pregnant mother.

10. A The fetal heart rate measuring assembly according to claim 1, wherein the fetal heart rate measuring assembly comprises a main housing connected to a harness adapted to be suspended about the neck of the pregnant mother, and that the main housing comprises a second display and the measurement presentation unit which is a first display.

11. The fetal heart rate measuring assembly according to claim 10, wherein the second display faces towards the face of the pregnant mother when the main housing is carried around the neck with the harness.

12. The fetal heart rate measuring assembly according to claim 10, wherein the first display faces away from the face of the pregnant mother when the main housing is carried around the neck with the harness.

13. The fetal heart rate measuring assembly according to claim 1, wherein the ultrasound transducer is arranged in a transducer housing and that a hydrogel film is arranged on a transducer housing contacting face.
